(19) United States
(12) Patent Application Publication
(10) Pub. No.: US 2011/0172783 A1
(43) Pub. Date: Jul. 14, 2011

(54) ONE-CYLINDER THRUST ROLL METHOD, DEVICE THEREFOR AND PRODUCTS MANUFACTURED THEREWITH

(75) Inventor: Anton Walliser, Basel (CH)

(73) Assignee: DUMONT SWITZERLAND AG, Zug (CH)

(21) Appl. No.: 12/865,705

(22) PCT Filed: Jan. 28, 2009
(86) PCT No.: PCT/CH2009/000033

§ 371 Date: Feb. 18, 2011

(30) Foreign Application Priority Data
Jan. 31, 2008 (CH) 141/08

(51) Int. Cl. A61F 2/30; C22C 38/02; C22C 38/18; C22C 38/04; B25B 9/02; B21B 37/00; B21H 7/00

(52) U.S. Cl. 623/23.41; 420/42; 420/34; 294/99.2; 72/12.2; 72/8.5; 623/18.11

(57) ABSTRACT

The invention relates to a single-piece metal strip having no weld seams and made of a polycrystalline metal, comprising at least one region in which the crystallites have a comparatively stronger anisotropic orientation, and at least one region in which the crystallites have a comparatively less strong anisotropic orientation, and wherein 0-20 X-ray diffractograms measured at two arbitrary points of the strip by way of CuKalpha radiation produce no statistically significant differences with respect to the position and shape of the respectively corresponding pikes, and to tweezers, supporting implants, and joint prostheses comprising said metal strip. The invention further relates to a roll method for obtaining said metal strip.

### REFERENCE TO EARLIER APPLICATIONS

This application claims the priority of Swiss patent application no. 141/08, of which the entire contents of disclosure are included herewith by reference.

### FIELD OF THE INVENTION

The present invention relates to the manufacture of precision instruments such as tweezers.

Such instruments have two legs, which are connected to one another at the one end, by spot welding for example. The other ends of the two legs are both free and are located with a small distance between them, and may resiliently be pressed together. In the case of a tweezers, for example, the free ends of the legs may be shaped into fine tips, which must fit together perfectly in order for the tweezers to perform its function properly. Since the two legs are joined together at the other end thereof only after their free tips have been produced, the tweezers must usually be aftertreated by hand to ensure the fit of the tips.

On the other hand, as is known, continuous metal parts such as rails, profiles or wires are produced using two-cylinder rolling mills. These exert a symmetrical squeezing force on the metal. The crystal lattice axes of the newly layered structure are oriented in a star formation extending from the middle towards both sides in the direction of rolling; a “roll ridge” is formed in front of the rolls. As a result, the rolled material is spread both widthwise and lengthwise. It is not possible to prevent the rolling material from spreading widthwise by providing lateral delimiters or openings in the rolling cylinders, because the laterally oriented forces in the spreading material would create an explosive effect with burr formation. This would lead to a breaking of the rolling cylinders or would cause the entire machine to break or seize up. In order to prevent the rolling material from spreading laterally in two-cylinder rolling mills, a traction device exerting a pull large enough to ensure that the rolling material can only undergo insignificant lateral spread must be provided behind the rolls.

### PRIOR ART

In paragraph 5 of EP 1 275 472 A it is mentioned that complex contours can be created by rolling with single-roll mechanical tools, wherein the rolling process takes place at an effective point between the roll and the tool surface.

WO 01/13756 A discloses a tweezers made of light metal which consists of a single part and has no welded spot. It is produced without rolling by separating an extruded light metal profile into a plurality of such tweezers.

The first objective of the present invention is to provide an intermediate product in the form of a metal strip having new material properties, which is suitable for manufacturing instruments of the kind described in the introduction. A further objective is to provide a process for producing such a metal strip and the associated device, wherein this process in particular produces in the metal strip the resilient parts of the legs of instruments such as those described in the introduction. A further objective of the invention is to provide a process for producing other metal objects that contain a resilient region or have complex profile shapes.

### SUMMARY OF THE INVENTION

The first objective is solved according to the invention by a single-piece metal strip having no weld seams and made of a polycrystalline metal, comprising at least one region in which the crystallites have a comparatively more pronounced anisotropic orientation, and at least one region in which the crystallites have a comparatively less pronounced anisotropic orientation; and wherein Θ-2Θ X-ray diffractograms measured at two arbitrary points of the strip using CuKα radiation do not show statistically significant differences with respect to the position and shape of the respectively corresponding pikes. The comparatively more pronounced anisotropic orientation of the crystallites in the one region is more pronounced than the comparatively less pronounced anisotropic orientation of the crystallites in the other region.

This region of comparatively more pronounced anisotropic crystallite orientation which is resilient is obtainable by a rolling process for deforming an initial shaped body of metal, wherein the rolling process is performed between a roll having an axis of rotation and a rolling surface on the one hand, and a support having a support surface on the other hand; characterized in that the angular velocity ω of the roll is controlled in such manner that

$$0 \leq ω < \frac{v}{R} , \qquad ( 1 )$$

applies for at least one point of the roll surface that contacts the initial shaped body in rolling manner, and in which formula ν is the rolling velocity and R is the distance between the axis of rotation and the described point on the roll surface measured perpendicularly to the axis of rotation of the roll.

Preferred embodiments of the metal strip according to the invention, of the production process and of the associated device are described in the dependent claims. The rolling device for performing the process according to the invention as well as tweezers obtainable using the process according to the invention, are likewise objects of the invention.

### DETAILED DESCRIPTION OF THE INVENTION

“Rolling velocity ν” in the above formula (1) is the speed at which an imaginary point on the axis of rotation of the roll, lying equidistantly between the points where the axis of rotation intersects the two frontal faces of the roll, moves relative to the initial shaped body before it enters the rolling zone between the roll and the support.

The process according to the invention is carried out in such manner that at least one point on the roll surface contacting the initial shaped body in rolling manner the angular velocity ω of the roll is less than ν/R, wherein ν and R have the meanings defined previously. This rolling point may be the point or points whose distance R from the axis of rotation is or are minimal compared to the distances R from the other rolling points on the rolling surface. It is preferred that for each rolling point of the rolling surface ω is smaller than ν/R, wherein ν and R have the meanings defined previously. These features differ from a conventional rolling process with two counter-rotating rolls, wherein for each rolling point of the roll surfaces of both rolls the angular velocity ω of the roll in question is greater than the quotient ν/R, wherein ν and R have the meanings defined previously.

The angular velocity ω is the angle (measured in radians) that the roll rotates per unit of time. The unit of the angular velocity ω is thus s⁻¹.

The angular velocity ω preferably has a value of 30 to 95%, more preferably of 50 to 80% of the quotient ν/R.

The reduced angular velocity ω in the rolling process according to the invention may be achieved as follows: The roll is pressed against the initial shaped body to be rolled and lying on the support with a normal force F sufficient to deform the initial shaped body. The roll is then pushed or pulled in this pressed state over the initial shaped body at a desired rolling speed ν. To ensure that the angular velocity ω remains less than ν/R as provided for in the invention, the rotation of the roll is simultaneously hindered or braked. As a result of the braked rotation of the roll in the process according to the invention the initial shaped body is not simply rolled flat, but a compressed bulge of material is formed in front of the roll, which is pushed ahead of the roll like a pressure wave.

This hindering or braking of the roll's rotation may be effected via a suitable braking device, which acts on the roll during the rolling process. On the other hand said material bulge also has a braking effect on the angular velocity ω of the roll. If the parameters of the rolling process, such as contact pressure, rolling speed and so on are selected appropriately, it may be possible in favourable situations to dispense with the explicit braking of the roll as soon as this material bulge is formed. If a brake is used, then it may be any known type of brake, for example a friction brake, such as a drum brake, a disc or wedge brake, an eddy current brake, or a brake that is operated or actuated hydraulically (viscosity brake). The angular velocity of the roll may also be controlled by providing an electric or hydraulic motor which by itself may drive the roll but whose speed is selected such as to enable the said control of the angular velocity of the roll. The braking force may be set or controlled with a suitable braking force adjuster. The minimum required braking force is defined such that the roll's rotation is braked until ω<ν/R is achieved at least one rolling point of the roll surface. On the other hand, however, the braking force is also large enough to stop the rotation of the roll partially or even completely (i.e. ω≧0). The braking force may be varied between these two threshold values, thus giving an angular velocity ω of the roll that is less than ν/R and equal to or greater than zero.

The magnitude of the pull (push) depends on the contact pressure of the roll against the initial shaped body, on the pressure wave created thereby in front of the roll, and on the rolling speed ν. The contact pressure must be consistent with the desired degree of forming of the rolling procedure, but it must be less than the pressure that would result in approaching or exceeding the yield strength of the material of the initial shaped body. The size of the braking material bulge that is pushed ahead of the roll depends directly on the magnitude of the rolling pressure. Its braking effect on the angular velocity ω and on the rolling speed of the roll, and thus also on the pull (push) may be increased by designing the roll as a profiled roll, which results in a greater degree of cold forming and thus requires from the roll a greater working effort. The rolling speed ν affects the height of the material bulge in conjunction with the other factors.

In the process according to the invention the roll may be guided over the initial shaped body to be rolled in a predefined linear or curved path according to the desired profile. In this context, the roll is preferably pushed according to the invention.

The process according to the invention is not a continuously operating process, since the support only has finite dimensions, and a rolling cycle ends at the latest after the roll has rolled over the entire support. On the other hand, this also offers the possibility of rolling an initial shaped body in several consecutive cycles, whereby these cycles may be performed one directly after the other, e.g. using the same roll every time, or with replacement of the roll between two consecutive cycles.

According to the invention, the roll does not have to be a cylindrical roll; it may also be a roll with a non-cylindrical shape. The axis of rotation of the roll, from which the distances R to the rolling points of the roll surface are measured, does not have to be located inside the roll; however, it is preferably inside the roll. These distances R are greater than zero, and in extreme cases may go towards infinity; the latter case corresponds to a roll with a flat roll surface. For the purposes of the invention, however, the roll is preferably a cylindrical roll, or a rotationally symmetrical profile roll, or a sector roll having a rolling surface that is cylindrical or has a rotationally symmetrical profile. In the case of a cylindrical roll, a rotationally symmetrical profile roll, or a sector roll having a rolling surface that is cylindrical or has a rotationally symmetrical profile, the roll's axis of symmetry is preferably coincident with the central axis of the roll. In the cases of a cylindrical roll, a sector roll, a rotationally symmetrical profile roll, or a sector roll having a surface with rotationally symmetrical rolling profile, the said distance between the axis of rotation and the rolling point of the roll surface is identical with the radius R of the roll at this point. The roll profile that may be optionally applied to the roll surface is preferably such that the baseline of its cross sectional profile corresponds to the strip width of the initial shaped body to be rolled (i.e. to the rolled region of the metal strip according to the invention). In order for a rolling process according to the invention to be carried out, i.e. with no more than negligible lateral spread, the cross-sectional area of the rolled metal strip preferably remains constant during the reshaping of its own profile. During the process according to the invention, the roll simultaneously performs two functions: 1) the pressing function to reduce the thickness of the initial shaped body, and 2) the pulling function to lengthen the rolled material, which corresponds to a reduction in the cross-section whereas the width thereof remains unchanged or increases only insignificantly.

The support may be flat but it may also have a surface that is suitably curved or profiled in three spatial dimensions. In this case, the initial shaped body then not only undergoes rolling deformation but also form rolling. If the surface is to be profiled, this profile is preferably also selected such that by itself it results in reshaping wherein the cross-section remains constant.

In the process according to the invention, explicit heating is preferably not done, so that the only heat source acting on the initial shaped body is the heat that is generated within the shaped body itself during rolling. This means preferably that the temperature of the initial shaped body and the resulting rolled metal strip during the entire rolling process does not exceed 100° C. at any point.

The initial shaped body that can be rolled by the process according to the invention may consist of any sufficiently ductile metal. If a metal strip according to the invention is to be produced, the initial shaped body also consists of a polycrystalline metal. Examples of such metals would be steel alloys, bronze, aluminium, copper, titanium, or brass. It is preferably a metal that crystallises according to a body-centred cubic (bcc) crystal lattice, that is to say a ferritic, martensitic, or mixed ferritic/martensitic steel. Among the martensitic or mixed ferritic/martensitic steels, the α′ modification of the martensite is preferred. Strictly, speaking, this crystallises in a body-centred tetragonal lattice, but for practical purposes it may also be considered to undergo body-centred cubic crystallisation. According to the invention, a martensitic steel that is free from nickel and molybdenum is preferred (in this context, “free” means having a content of less than 0.01 percent by weight). More preferably, it has the composition Cr 12.50-14.50 percent by weight, C 0.42-0.50 percent by weight, Si max. 1.00 percent by weight, Mn max. 1.00 percent by weight, P max. 0.045 percent by weight, S max. 0.030 percent by weight, the rest essentially consisting of iron and unavoidable contaminants. In particular, it may be a steel corresponding to material number 1.4034. The initial shaped body is preferably already in the form of a metal strip or of sheet metal.

To enable the rolling deformation to take place more easily and with lower pressures, the metallic initial shaped body, if consisting of a steel alloy, may undergo a beforehand heat treatment, preferably with subsequent quenching in cold water. This treatment reduces the metal's strength isotropically. In the field of steel alloys, this thermal treatment is known as solution heat treatment, and typically a temperature range from 1050-1080° C. is selected, the heat treatment typically lasts from about 10 minutes to about 1 hour, preferably half an hour.

The method according to the invention is suitable for production all types of tweezers, needle holders, springs, dissectors, clamps, scissors (for example hairdressing scissors), knives (in which case rolling may be performed along the axis of the blade instead of transversally to the blade axis, as has been done so far), or special profiles of any kind (for example for constructing façades or pipes). It is particularly suitable for producing parts that have at least one resilient region, thus for instance for manufacturing tweezers, needle holders or springs, and especially also for tweezers with an additional forward- or backward-acting cutting function (tweezer scissors), or for surgical or orthopaedic implants.

The device according to the invention, with which the process according to the invention is performed, includes at least one roll, one support, and one brake as described for exemplary purposes in the preceding, which is capable of braking the angular velocity ω of the roll during rolling. The support itself may be movable or non-movable, it is preferably non-movable. The device according to the invention further includes the bearings and associated guides that are necessary to press the roll against the material, and to move it (push or pull). These roll guides preferably include hydraulic cylinders or mechanical systems which are capable of guiding the roll over the support accurately and with a constant or controlled distance curve (the thickness of the resulting rolled metal strip is then constant or variable, respectively). These hydraulic or mechanical roll guides are known in the field of rolling and do not need to be described further. By the appropriately controlled braking of the roll and the simultaneous forward pulling or pushing thereof an additional drawing device for the rolled material is unnecessary in the device according to the invention.

The result of the process according to the invention and other, optional prior shaping steps, is an at least partly rolled metal strip. This metal strip does not necessarily have a regular or flat shape. It consists of one single piece, i.e. it does not consist of two or more separate parts that have been screwed, riveted or stuck together. It also has no weld points.

In general, a strip according to the invention consists of a polycrystalline metal that includes a rolled region such as is described in the preceding, in which region the anisotropic orientation of the crystallites is comparatively more pronounced. In the region or regions that is(are) not rolled, it retains the crystallite orientation of the initial shaped body, that is to say the orientation of the crystallites is comparatively less pronounced, or in some cases may be essentially isotropic. The term “not rolled” also refers to a pretreatment of the initial shaped body under rolls, provided that the last step of such pretreatment has been a heat treatment such as is described in the preceding, and which undoes any changes in the lattice that may have been caused by the rolling.

The orientation of the crystallites is defined for the purpose of the present invention in terms of the orientation density function, also known in the technical community by the abbreviation ODF. In English it is called the “orientational distribution function”, which is also abbreviated as ODF.

For the purposes of the present application, an orthogonal coordinate system is used for the rolled strip, the X-axis of which extends parallel to the rolling direction of the strip region rolled according to the invention; the Y-axis of which is perpendicular to the rolling direction, and extends to the left when the strip is viewed from above, and the Z-axis of which is the normal as the cross product of the X-axis and the Z-axis.

Said orientational distribution function ODF is determined within the scope of the present application from X-ray structural data obtained with CuKα radiation (λ=1.54 Angström). On the one hand, for example, disc-shaped samples having a flat surface to be irradiated may be cut out of the strip to be examined. In this case, the sample to be examined is cut out of the strip in such manner that the surface to be irradiated stands vertically on the Z-axis. If the strip already has a flat surface that stands vertically on the Z-axis, the strip might also be measured directly at this surface.

This ODF may be defined on the one hand as

$$f ( ϕ 1 , Φ , ϕ 2 ) = \frac{d V}{d Φ d ϕ1 d ϕ2sin Φ} \qquad ( 2 a )$$

In formula (2a),
φ₁, Φ, φ₂ stand for the three Euler angles that describe the rotation of the internal crystallite coordinate system relative to the sample coordinate system. The internal crystallite coordinate system is individual for each crystallite;
dV/dΦdφ₁dφ₂ stands for the differential volume fraction dV of those crystallites in which the internal crystallite coordinate system has an orientation within a differential, given spatial angle fraction (described by the differential Euler angles dΦ, dφ₁, dφ₂);
V: stands for the total volume of all irradiated crystallites.

This ODF may be calculated as f(φ₁, Φ, φ₂) from pole figure measurements. The pole figures and the ODF are approximated as series expansions of generalised spherical harmonic functions, these two approximations are, inserted in the fundamental equation of the texture analysis, and the coefficients of the series expansion are calculated from that. This method is described in sections 11.4.1 and 11.6.5 (“Harmonic method”) of the textbook “Moderne Röntgenbeugung” (Modern X-ray diffraction), L. Spiess, R. Schwarzer, H. Behnken, G. Teichert, October 2005, B. G. Teubner Verlag, Wiesbaden, Germany. This description is included in this document by reference.

Alternatively, the ODF may also be defined with formula (2b):

$$W ( ϑ , ϕ ) ≡ \frac{d V}{d Ω} \frac{4 π}{V} \qquad ( 2 b )$$

In formula (2b),
dV/dΩ: stands for the differential volume fraction dV of those crystallites in which the area normals of the irradiated surface of the sample (see below) fall within a differential, given spatial angle fraction dΩ in the internal crystallite coordinate system (refer to formula (2a) above for a description thereof).
V: stands for the total volume of all irradiated crystallites.

This orientational distribution function W(θ,φ) includes a polar angle θ measured from the z axes of the internal crystallite coordinate systems and an azimuthal angle φ measured from the x-axes as variables. This ODF is obtained by the following steps, a)-c) (see also J. Appli. Cryst. 1970, 3, p. 313ff.):

a) The sample is fixed on the sample holder of the diffractogram such that the surface normal of the surface to be irradiated is perpendicular to the axis of the diffractometer. The goniometer angle 2θ is selected such that a diffraction at a crystal lattice plane family with a given Miller index (hkl) is detected. The sample (or the strip itself) is then inclined such that the surface normal of the surface to be irradiated is rotated by an angle α from the normal of this plane family towards the diffractometer axis. With this inclination, and simultaneous rotation of the sample through 360° about the surface normal of the surface to be irradiated while retaining the goniometer angle 2θ selected in advance, the cumulative diffraction intensity Iₕₖₗ(α) is measured. This measurement is carried out for a total K of different angles α, but always with the same θ.
b) Each Iₕₖₗ(α) determined in a) is assumed to be representable by a series expansion of the form

$${I}_{hkl} ( α ) = A ( h , k , l , θ , λ ) ( 1 + {\sum }_{v}^{V} {\sum }_{w}^{W} {C}_{vw} {K}_{vw} ( {ϑ}_{hkl} , {ϕ}_{hkl} ) {P}_{v} ( cos α ) ) \qquad ( 3 )$$

In this equation, K_{vw}(θₕₖₗ,φₕₖₗ) is the value of the “symmetry-adapted spherical harmonic” (SASH) K_{vw} adapted to the crystal lattice symmetry of the metal in question at the angle pair (θ,φ) describing the direction of the normal vector of the crystal plane family having Miller Index (hkl) in the internal crystal coordinate systems. The index v only runs over the even numbers greater than zero up to the maximum considered number V. The larger the value of V, the higher the accuracy. The index w runs over all such linearly independent spherical harmonics of order v. The number K of Iₕₖₗ(α) measured in a) must be one greater than the total number of summands in the double sum of formula (3). Pᵥ(cos α) is the value of the Legendre polynomial of order v at cos α. Formula (3) also includes:

$$A ( h , k , l , θ , λ ) = \frac{Q}{2 μ} {\vert {\sum }_{j = 1}^{N} {f}_{j} ( \frac{sin ( θ )}{λ} ) exp ( - 2 iπ ( {hx}_{j} + {ky}_{j} + {lz}_{j} ) ) \vert}^{2} \frac{{P}_{hkl} ( 1 + {cos}^{2} ( 2 θ ) )}{8 {sin}^{2} ( θ ) cos ( θ )} exp ( B \frac{{sin}^{2} ( θ )}{{λ}^{2}} ) \qquad ( 4 )$$

wherein:
Q: represents a constant that is the same for all reflections;
μ: represents the linear coefficient of absorption of the metal under investigation for CuKα irradiation, these coefficients of absorption are known;

$${f}_{j} ( {f}_{j} ( \frac{sin ( θ )}{λ} ) \text{?} ) : \\ \text{?} \text{indicates text missing or illegible when filed}$$

represents the atomic form factor of the j-th atom in the unit cell depending on sin(θ)/λ. λ is 1.54 Angström. These atomic form factors are known. The sum in which these atomic form factors occur runs over all N atoms in the unit cell;
xⱼ, yⱼ, zⱼ: represent the coordinates of the j-th atom in the unit cell in internal crystallite coordinates (see above).
Pₕₖₗ: Represents the multiplicity of the detected intensity, i.e. the number of equivalent crystal plane families that contribute to Iₕₖₗ(α). These multiplicities are known for all types of crystal lattices.
B: Represents the temperature factor, which is assumed to be approximately isotropic and the same for all atoms of the unit cell, These temperature factors are known.

From equations (3) the coefficients C_{vw} and also Q are determined in turn.

c) The orientational distribution function W(θ,φ) is obtained via the coefficient C_{vw} contained in b) by using formula

$$W ( ϑ , ϕ ) = 1 + {\sum }_{v}^{V} {\sum }_{w}^{W} {C}_{vw} {K}_{vw} ( ϑ , ϕ ) \qquad ( 5 )$$

wherein K_{vw}(θ,φ) are again the symmetry-adapted spherical harmonics discussed above, the angle pair (θ,φ) within the internal crystallite coordinate system is as described above, and v, V, w and W have the meanings defined above.

The orientation of the crystallites in the region rolled according to the invention is more strongly anisotropic than in another region that has not been rolled or has been rolled conventionally. The anisotropy of the crystallite orientation in the region where the said anisotropy is more pronounced, is preferably such that the ODF described in the preceding formula (2b) contains at least one C_{vw} in its approximate series expansion that is of a magnitude of at least 0.050; this C_{vw} is particularly preferably of a magnitude of at least 0.100, and especially preferably of at least 0.200. On the other hand, the ODF described according to formula (2b) above in the region with comparatively less pronounced anisotropic crystallite orientation, is preferably such that in the said ODF series expansion is such that none of the C_{vw} is of a magnitude of greater than 0.050, i.e. it is essentially isotropic (for purely isotropic crystallite orientation, all C_{vw}, would be zero).

In the case of strips that include both a region that has been rolled according to the invention and a region that has not been rolled, and which are made from a ferritic, martensitic, or mixed ferritic/martensitic steel as described above, this anisotropy of orientation is expressed particularly as follows: If θ-2θ diffractograms are recorded of samples of the rolled and not rolled regions of such a strip such that the axis of the diffractometer is parallel to the surface of the sample to be irradiated, it is found that the crystallites in the region that has been rolled according to the invention are comparatively more frequently orientated so that their plane family having Miller Index (200) is parallel, to the irradiated surface. To a lesser extent, the crystallites are also orientated such that their plane family having Miller Index (211) is more frequently parallel to the irradiated surface. This special orientation of the crystallites becomes more pronounced as one progresses from the edge zones of the region that has been rolled according to the invention (from the outside) towards the middle (towards the inside). In contrast, on a strip made from the same metal, but which includes a region that has been rolled according to a conventional method (with two rolls), no such special orientation is noted.

Also in general terms, the following is observed in strips that are made of a polycrystalline metal, preferably of a metal that crystallises in a body-centred cubic (bcc) crystal lattice, particularly preferably of a ferritic, martensitic, or mixed ferritic/martensitic steel as described above (in particular corresponding to material no. 1.4034), and which include a region that has been rolled according to the invention and a region that has not been rolled: When they are examined at two arbitrary points in accordance with the diffraction method described in the previous paragraph, no statistically significant differences are found in the position or shape of their pikes if the corresponding pikes of the two diffractograms are compared with each other, i.e. the two pikes never differ from one another to a statistically significant degree in terms of the position and shape of their pikes. This again is different from a strip that is made of the same metal but includes a region that has been rolled according to a conventional method (with two rolls): in that case, the pike from a diffractogram of the rolled region may be shifted to a statistically significant degree and/or its shape may be have been distorted to a statistically significant degree with respect to the corresponding pike from a diffractogram of the region that has not been rolled.

For the purposes of the present application, the “shape” of a pike is understood to be the symmetry of the pike with respect to its maximum and the sharpness thereof (ratio of half-width to maximum intensity). The intensity of the pike is not understood to be the “shape” of the pike.

In order to determine whether two pikes differ from one another “to a statistically significant degree in terms of their position and shape”, the following steps 1)-4) are carried out for the purposes of the present application:

1) The two diffractograms for comparison are provided in a form in which the absolute intensity curves of the two pikes for comparison have been plotted as a function of 2θ in discrete counting intervals having width 0.05°. Each of these counting intervals will be identified in the following by an associated Index i.
2) In each of the two diffractograms the largest possible contiguous, matching 2θ region is searched which contains the maxima of the two pikes to be compared, and in which always either the absolute intensity of the i-th counting interval from the first diffractogram is at least twice as large as the associated baseline, or the absolute intensity of the corresponding i-th counting interval of the second diffractogram is at least twice as large as the associated baseline, or even both i-th intensities are each at least twice as large as the associated respective baseline. If no such region exists, the two pikes are considered to be “differ from one another to a statistically significant degree with regard to position”, and the remainder of the test is not carried out anymore.
3) However, if such a region having a total of k contiguous counting intervals does exist, the characteristic chi-square is calculated from all these counting intervals:

$${x}^{2} = {\sum }_{i = 1}^{k} \frac{{( {}_{1}^{}{b}{}_{i}^{} - {}_{2}^{}{b}{}_{i}^{} )}^{2}}{2 ( {}_{1}^{}{b}{}_{i}^{} + {}_{2}^{}{b}{}_{i}^{} )} \qquad ( 6 )$$

In this formula, ₁bᵢ and ₂bᵢ are the intensities of the first and second pikes, respectively, in the i-th counting interval, their base-line intensity having been removed and having been normalised to a pike maximum of 100 (one hundred) counts. This normalisation of the pikes to be compared to a maximum of 100 counts is done, firstly, because the number of irradiated crystallites is not constant from one sampling site to another (this results in differing intensities in the diffractograms taken at the two sampling sites), and, secondly, because differences may exist in the anisotropy of the crystallite orientation (this results in variations in the intensity ratios of the pikes within the same diffractogram; at least one region with relatively more pronounced anisotropic crystallite orientation and at least one region with relatively less pronounced anisotropic crystallite orientation is is in particular required in the case of a strip according to the invention). The statistical test described here however should only show, without consideration of and independently from the anisotropy of the crystallite's orientation, statistically significant differences in the properties of the crystallites themselves, e.g. a lower average size (visible as a widening of the pike), or tensions within the crystal lattice (visible as a shift of the pike maximum, pike broadening or asymmetry in the pike shape). The above formula (6) is derivable from the formula known in mathematics for the Chi-Square test variable for observed counting intensities ₁bᵢ or ₂bᵢ, if one takes the average from ₁bᵢ and ₂bᵢ, μᵢ, as the associated i-th expectation value, and the square root of this average value as the standard deviation σᵢ.

4) The Chi-Square characteristic calculated in step 3) is compared with the value of the Chi-Square distribution for k degrees of freedom according to the following table 1 with a significance threshold of 0.001% (k is the number of counting intervals of the contiguous region identified in step 2):

TABLE 1

| Degrees | | | | | | |
|---|---|---|---|---|---|---|
| of | | | | | | |
| freedom | Significance threshold | | | | | |
| k | 0.001% | 0.01% | 0.1% | 1% | 5% | 10% |
| 2 | 23.03 | 18.42 | 13.82 | 9.21 | 5.99 | 4.61 |
| 3 | 25.75 | 20.97 | 16.14 | 11.24 | 7.74 | 6.19 |
| 4 | 28.47 | 23.51 | 18.47 | 13.28 | 9.49 | 7.78 |
| 5 | 30.79 | 25.68 | 20.46 | 15.04 | 11.04 | 9.21 |
| 6 | 33.11 | 27.86 | 22.46 | 16.81 | 12.59 | 10.64 |
| 7 | 35.22 | 29.84 | 24.29 | 18.45 | 14.05 | 12.00 |
| 8 | 37.33 | 31.83 | 26.12 | 20.09 | 15.51 | 13.36 |
| 9 | 39.31 | 33.70 | 27.86 | 21.65 | 16.91 | 14.67 |
| 10 | 41.30 | 35.56 | 29.59 | 23.21 | 18.31 | 15.99 |
| 11 | 43.19 | 37.35 | 31.25 | 24.71 | 19.67 | 17.27 |
| 12 | 45.08 | 39.13 | 32.91 | 26.22 | 21.03 | 18.55 |
| 13 | 46.90 | 40.86 | 34.52 | 27.68 | 22.36 | 19.81 |
| 14 | 48.72 | 42.58 | 36.12 | 29.14 | 23.68 | 21.06 |
| 15 | 50.48 | 44.25 | 37.69 | 30.57 | 24.99 | 22.30 |
| 16 | 52.24 | 45.92 | 39.25 | 32.00 | 26.30 | 23.54 |
| 17 | 53.96 | 47.56 | 40.78 | 33.40 | 27.58 | 24.77 |
| 18 | 55.68 | 49.19 | 42.31 | 34.81 | 28.87 | 25.99 |
| 19 | 57.36 | 50.79 | 43.81 | 36.19 | 30.14 | 27.30 |
| 20 | 59.04 | 52.39 | 45.31 | 37.57 | 31.41 | 28.41 |
| 21 | 60.69 | 53.96 | 46.79 | 38.93 | 32.67 | 29.61 |
| 22 | 62.34 | 55.52 | 48.27 | 40.29 | 33.92 | 30.81 |
| 23 | 63.96 | 57.07 | 49.72 | 41.63 | 35.17 | 32.00 |
| 24 | 65.58 | 58.61 | 51.18 | 42.98 | 36.42 | 33.20 |
| 25 | 67.18 | 60.14 | 52.62 | 44.31 | 37.65 | 34.38 |
| 26 | 68.77 | 61.66 | 54.05 | 45.64 | 38.89 | 35.56 |
| 27 | 70.34 | 63.16 | 55.47 | 46.96 | 40.11 | 36.74 |
| 28 | 71.92 | 64.66 | 56.89 | 48.28 | 41.34 | 37.92 |
| 29 | 73.47 | 66.15 | 58.30 | 49.59 | 42.56 | 39.09 |
| 30 | 75.02 | 67.63 | 59.70 | 50.89 | 43.77 | 40.26 |
| 31 | 76.56 | 69.10 | 61.10 | 52.19 | 44.98 | 41.42 |
| 32 | 78.09 | 70.57 | 62.49 | 53.49 | 46.19 | 42.58 |
| 33 | 79.61 | 72.03 | 63.87 | 54.77 | 47.70 | 43.74 |
| 34 | 81.13 | 73.48 | 65.25 | 56.06 | 48.60 | 44.90 |
| 35 | 82.64 | 74.92 | 66.62 | 57.34 | 49.80 | 46.06 |
| 36 | 84.14 | 76.36 | 67.99 | 58.62 | 51.00 | 47.21 |
| 37 | 85.63 | 77.79 | 69.34 | 59.89 | 52.19 | 48.36 |
| 38 | 87.12 | 79.22 | 70.70 | 61.16 | 53.38 | 49.51 |
| 39 | 88.60 | 80.64 | 72.05 | 62.43 | 54.57 | 50.66 |
| 40 | 90.08 | 82.06 | 73.40 | 63.69 | 55.76 | 51.81 |
| 41 | 91.55 | 83.47 | 74.74 | 64.95 | 56.94 | 52.95 |
| 42 | 93.01 | 84.88 | 76.08 | 66.21 | 58.12 | 54.09 |
| 43 | 94.47 | 86.28 | 77.42 | 67.46 | 59.30 | 55.23 |
| 44 | 95.92 | 87.68 | 78.75 | 68.71 | 60.48 | 56.37 |
| 45 | 97.37 | 89.07 | 80.07 | 69.96 | 61.66 | 57.50 |
| 46 | 98.81 | 90.46 | 81.40 | 71.20 | 62.83 | 58.64 |
| 47 | 100.25 | 91.84 | 82.72 | 72.44 | 64.00 | 59.77 |
| 48 | 101.69 | 93.22 | 84.04 | 73.68 | 65.17 | 60.91 |
| 49 | 103.11 | 94.59 | 85.35 | 74.92 | 66.34 | 62.04 |
| 50 | 104.54 | 95.97 | 86.66 | 76.15 | 67.50 | 63.17 |
| 51 | 105.96 | 97.34 | 87.97 | 77.38 | 68.67 | 64.29 |
| 52 | 107.38 | 98.70 | 89.27 | 78.62 | 69.83 | 65.42 |
| 53 | 108.79 | 100.06 | 90.57 | 79.84 | 70.99 | 66.55 |
| 54 | 110.20 | 101.42 | 91.87 | 81.07 | 72.15 | 67.67 |
| 55 | 111.61 | 102.77 | 93.17 | 82.29 | 73.31 | 68.80 |
| 56 | 113.01 | 104.13 | 94.46 | 83.51 | 74.47 | 69.92 |
| 57 | 114.41 | 105.47 | 99.75 | 84.73 | 75.62 | 71.04 |
| 58 | 115.80 | 106.82 | 97.04 | 85.95 | 76.78 | 72.16 |
| 59 | 117.19 | 108.16 | 98.32 | 87.16 | 77.93 | 73.28 |
| 60 | 118.58 | 109.50 | 99.61 | 88.38 | 79.08 | 74.40 |
| 61 | 119.96 | 110.84 | 100.89 | 89.59 | 80.23 | 75.51 |
| 62 | 121.35 | 112.17 | 102.17 | 90.80 | 81.38 | 76.63 |
| 63 | 122.73 | 113.50 | 103.44 | 92.01 | 82.53 | 77.74 |
| 64 | 124.10 | 114.83 | 104.72 | 93.22 | 83.68 | 78.86 |
| 65 | 125.47 | 116.16 | 105.99 | 94.42 | 84.82 | 79.97 |
| 66 | 126.85 | 117.48 | 107.26 | 95.63 | 85.96 | 81.09 |
| 67 | 128.21 | 118.80 | 108.52 | 96.83 | 87.11 | 82.20 |
| 68 | 129.58 | 120.12 | 109.79 | 98.03 | 88.25 | 83.31 |
| 69 | 130.94 | 121.44 | 111.05 | 99.23 | 89.39 | 84.42 |
| 70 | 132.30 | 122.75 | 112.32 | 100.43 | 90.53 | 85.53 |
| 71 | 133.66 | 124.07 | 113.58 | 101.62 | 91.67 | 86.64 |
| 72 | 135.01 | 125.38 | 114.84 | 102.82 | 92.81 | 87.74 |
| 73 | 136.36 | 126.68 | 116.09 | 104.01 | 93.94 | 88.85 |
| 74 | 137.71 | 127.99 | 117.35 | 105.20 | 95.08 | 89.96 |
| 75 | 139.06 | 129.29 | 118.60 | 106.39 | 96.22 | 91.06 |
| 76 | 140.41 | 130.60 | 119.85 | 107.58 | 97.35 | 92.17 |
| 77 | 141.75 | 131.89 | 121.10 | 108.77 | 98.48 | 93.27 |
| 78 | 143.09 | 133.19 | 122.35 | 109.96 | 99.62 | 94.37 |
| 79 | 144.43 | 134.49 | 123.59 | 111.14 | 100.75 | 95.48 |
| 80 | 145.76 | 135.78 | 124.84 | 112.33 | 101.88 | 96.58 |
| 81 | 147.10 | 137.07 | 126.08 | 113.51 | 103.01 | 97.68 |
| 82 | 148.43 | 138.37 | 127.32 | 114.69 | 104.14 | 98.78 |
| 83 | 149.76 | 139.65 | 128.56 | 115.88 | 105.27 | 99.88 |
| 84 | 151.09 | 140.94 | 129.80 | 117.06 | 106.39 | 100.98 |
| 85 | 152.41 | 142.22 | 131.04 | 118.24 | 107.52 | 102.08 |
| 86 | 153.74 | 143.51 | 132.28 | 119.41 | 108.65 | 103.18 |
| 87 | 155.06 | 144.79 | 133.51 | 120.59 | 109.77 | 104.27 |
| 88 | 156.38 | 146.07 | 134.75 | 121.77 | 110.90 | 105.37 |
| 89 | 157.70 | 147.35 | 135.98 | 122.94 | 112.02 | 106.47 |
| 90 | 159.02 | 148.63 | 137.21 | 124.12 | 113.15 | 107.57 |
| 91 | 160.33 | 149.90 | 138.44 | 125.29 | 114.27 | 108.66 |
| 92 | 161.65 | 151.18 | 139.67 | 126.46 | 115.39 | 109.76 |
| 93 | 162.96 | 152.45 | 140.89 | 127.63 | 116.51 | 110.85 |
| 94 | 164.27 | 153.72 | 142.12 | 128.80 | 117.63 | 111.94 |
| 95 | 165.58 | 154.99 | 143.34 | 129.97 | 118.75 | 113.04 |
| 96 | 166.89 | 156.26 | 144.57 | 131.14 | 119.87 | 114.13 |
| 97 | 168.19 | 157.53 | 145.79 | 132.31 | 120.99 | 115.22 |
| 98 | 169.50 | 158.79 | 147.01 | 133.48 | 122.11 | 116.32 |
| 99 | 170.80 | 160.06 | 148.23 | 134.64 | 123.22 | 117.41 |
| 100 | 172.10 | 161.32 | 149.45 | 135.81 | 124.34 | 118.50 |

If the Chi-Square characteristic calculated in step 3) is greater than the value listed in table 1 for the applicable number of degrees of freedom k, the two pikes are “different from each other to a statistically significant degree with regard to either position or shape”, otherwise they are “not different from each other to a statistically significant degree with regard to position and shape”.

This Chi-Square characteristic from two corresponding pikes from two diffractograms, from two arbitrary sites of strip that has been partly rolled according to the invention, but of which no part has been rolled conventionally, is preferably always so small that the two pikes may still be evaluated as “not different from each other to a statistically significant degree with regard to position and shape” even if the values used from the table are not those for the significance threshold 0.001%, but (in increasing levels of preference) for 0.01%, 0.1%, 1%, 5% or 10%.

In the case of a metal that crystallises according to a body-centred, particularly a body-centred cubic (bcc) crystal lattice (including the preferred ferritic, martensitic, or mixed ferritic/martensitic steels), or according to a surface centred lattice (for example the austenitic steels) statistically significant differences are most readily observed between the two diffractograms when the pikes with Miller. Index (200) are compared.

For example, diffractograms from two strips A and B from a steel with material number 1.4034, 70 mm long and 10 mm wide, and of which one end had not been rolled and had a constant thickness of about 1.5 mm, and of which the other end had been rolled either according to the invention (strip A) or conventionally (strip B) to a constant thickness of about 0.85 mm, were examined with a computer program according to the statistical test described above. The pike at 2θ approximately 64.8° (the (200) pike) was tested for statistically significant differences. The program required manual input of the positions of the pike maxima, and of baseline regions to the left and right of both pikes. As the baseline intensities the software calculated the averages of all intensities from both of these baseline regions. The program then identified the largest possible contiguous 2θ area in accordance with step 2) above, and the number of degrees of freedom k, and then calculated the Chi-Square value using formula (6) above. The following Chi-Square values and associated degrees of freedom k were obtained:

TABLE 2a
(Strip A)

| | | | | Are the pikes at |
|---|---|---|---|---|
| | | | | 64.8° from the 1^{st} |
| | | | | and 2^{nd} |
| | | | | diffractograms |
| | | | | “statistically |
| | | | | significantly |
| | | | | different from |
| | | | | each other in |
| | | Chi-Square | Degrees of freedom | terms of position |
| | | according to | (= number of | or shape” given a |
| | | formula (6) (four | counting intervals | significance |
| Measurement site | Measurement site | calculations with | considered of the | threshold of |
| of the 1^{st} | of the 2^{nd} | variously selected | contiguous 2θ | 0.001%? |
| diffractogram | diffractogram | baseline | area) | Yes/No |
| Back of the rolled | Back of the rolled | 0.014 | 17 | No |
| region, in | region, in | 0.003 | 17 | |
| direction of | direction of | 0.008 | 17 | |
| rolling | rolling | 0.028 | 17 | |
| Back of the rolled | Top of the rolled | 6.466 | 18 | No |
| region, in | region, in | 6.712 | 18 | |
| direction of | direction of | 6.448 | 18 | |
| rolling | rolling | 6.554 | 18 | |
| Back of the rolled | Top of the not | 2.924 | 18 | No |
| region, in | rolled region, in | 3.133 | 18 | |
| direction of | direction of | 2.985 | 18 | |
| rolling | rolling | 3.104 | 18 | |
| Back of the rolled | Back of the rolled | 6.865 | 23 | No |
| region, in | region, | 7.416 | 23 | |
| direction of | transversely to | 6.876 | 23 | |
| rolling | the direction of | 7.648 | 23 | |
| | rolling, outermost | | | |
| | edge area | | | |
| Back of the rolled | Back of the rolled | 6.325 | 25 | No |
| region, in | region, | 6.551 | 25 | |
| direction of | transversely to | 6.685 | 25 | |
| rolling | the direction of | 6.453 | 25 | |
| | rolling, inner | | | |
| | edge area | | | |
| Back of the rolled | Back of the rolled | 11.196 | 27 | No |
| region, in | region, | 11.200 | 27 | |
| direction of | transversely to | 11.188 | 27 | |
| rolling | the direction of | 11.192 | 27 | |
| | rolling, middle | | | |
| Top of the not | Top of the rolled | 4.067 | 18 | No |
| rolled region, in | region, in | 4.068 | 18 | |
| direction of | direction of | 4.086 | 18 | |
| rolling | rolling | 4.104 | 18 | |
| Top of the not | Back of the rolled | 3.449 | 21 | No |
| rolled region, in | region, | 4.634 | 23 | |
| direction of | transversely to | 5.016 | 23 | |
| rolling | the direction of | 5.088 | 23 | |
| | rolling, outermost | | | |
| | edge area | | | |
| Top of the not | Back of the rolled | 6.585 | 25 | No |
| rolled region, in | region, | 6.600 | 25 | |
| direction of | transversely to | 6.608 | 25 | |
| rolling | the direction of | 6.577 | 25 | |
| | rolling, inner | | | |
| | edge area | | | |
| Top of the not | Back of the rolled | 15.719 | 27 | No |
| rolled region, in | region, | 15.731 | 27 | |
| direction of | transversely to | 15.713 | 27 | |
| rolling | the direction of | 15.713 | 27 | |
| | rolling, middle of | | | |
| | the strip | | | |
| Back of the rolled | Top of the rolled | 6.162 | 23 | No |
| region, in | region, | 5.972 | 23 | |
| direction of | transversely to | 6.087 | 23 | |
| rolling | the direction of | 6.490 | 23 | |
| | rolling, outermost | | | |
| | edge area | | | |
| Back of the rolled | Top of the rolled | 11.851 | 28 | No |
| region, in | region, | 11.991 | 27 | |
| direction of | transversely to | 12.010 | 27 | |
| rolling | the direction of | 11.922 | 27 | |
| | rolling, inner | | | |
| | edge area | | | |
| Top of the not | Top of the rolled | 11.851 | 27 | No |
| rolled region, in | region, | 11.991 | 27 | |
| direction of | transversely to | 12.010 | 27 | |
| rolling | the direction of | 11.922 | 27 | |
| | rolling, middle of | | | |
| | the strip | | | |

TABLE 2b
(Strip B)

| | | | | Are the pikes at |
|---|---|---|---|---|
| | | | | 64.8° from the 1^{st} |
| | | | | and 2^{nd} |
| | | | | diffractograms |
| | | | | “statistically |
| | | | | significantly |
| | | | | different from |
| | | | Degrees of | each other in |
| | | Chi-Square | freedom (= number | terms of position |
| | | according to | of counting | or shape” given a |
| | | formula (6) (four | intervals | significance |
| Measurement site | Measurement site | calculations with | considered of the | threshold of |
| of the 1^{st} | of the 2^{nd} | variously selected | contiguous 2θ | 0.001%? |
| diffractogram | diffractogram | baseline | area) | Yes/No |
| Top of the rolled | Top of the rolled | 0.082 | 23 | No |
| region, | region, | 0.011 | 22 | |
| transversely to | transversely to | 0.001 | 22 | |
| the direction of | the direction of | 0.009 | 22 | |
| rolling, middle of | rolling, middle of | | | |
| the strip | the strip | | | |
| Top of the rolled | Top of the rolled | 4.029 | 24 | No |
| region, | region, | 4.239 | 24 | |
| transversely to | transversely to | 3.989 | 24 | |
| the direction of | the direction of | 4.188 | 24 | |
| rolling, middle of | rolling, outermost | | | |
| the strip | edge area | | | |
| Top of the rolled | Top of the rolled | 32.740 | 26 | No |
| region, | region, | 33.113 | 26 | |
| transversely to | transversely to | 33.228 | 26 | |
| the direction of | the direction of | 33.294 | 26 | |
| rolling, middle of | rolling, inner | | | |
| the strip | edge area | | | |
| Top of the rolled | Top of the rolled | 12.427 | 22 | No |
| region, | region, in | 12.600 | 22 | |
| transversely to | direction of | 12.414 | 22 | |
| the direction of | rolling | 12.413 | 22 | |
| rolling, middle of | | | | |
| the strip | | | | |
| Top of the not | Top of the rolled | 122.494 | 24 | Yes |
| rolled region, in | region, | 122.327 | 24 | |
| direction of | transversely to | 122.538 | 24 | |
| rolling | the direction of | 123.103 | 24 | |
| | rolling, outermost | | | |
| | edge area | | | |
| Top of the not | Top of the rolled | 112.289 | 26 | Yes |
| rolled region, in | region, | 112.319 | 26 | |
| direction of | transversely to | 112.514 | 26 | |
| rolling | the direction of | 114.382 | 27 | |
| | rolling, inner | | | |
| | edge area | | | |
| Top of the not | Top of the rolled | 138.629 | 24 | Yes |
| rolled region, in | region, | 137.743 | 23 | |
| direction of | transversely to | 138.966 | 24 | |
| rolling | the direction of | 138.993 | 24 | |
| | rolling, middle of | | | |
| | the strip | | | |
| Top of the not | Top of the rolled | 81.006 | 19 | Yes |
| rolled region, in | region, in | 80.696 | 19 | |
| direction of | direction of | 87.205 | 20 | |
| rolling | rolling | 87.221 | 20 | |

It can be seen that the conventionally rolled strip B shows pikes at 64.8° that “differ to a statistically significant degree in terms of either position or shape” when diffractograms of the not rolled and rolled areas are compared with each other. In contrast, no such statistically significant differences are observed for strip A, which was rolled according to the invention, regardless of the measurement sites of the two compared diffractograms. The same result is also obtained if any of the other significance thresholds from table 1 is chosen.

In the strip according to the invention, the region with relatively more pronounced anisotropic crystallite orientation preferably also has a less homogeneous microstructure than the region of relatively less pronounced anisotropic crystallite orientation, which is also caused by the rolling method according to the invention. These differences in homogeneity of the microstructures may be observed directly by comparing microphotographs of sections of the strip material from the regions in question.

At the same time, the rolling method according to the invention does not cause any internal stresses in the material, which may be recognized by the fact that the rolled region or regions do not show any tendency to warp during subsequent machining steps. Like any body, this metal strip has three principal axes of inertia. Since the metal strip according to the invention is rather elongated, the moment of inertia associated with one of the three principal axes of inertia is smaller than the two moments of inertia associated with the other two axes. This smallest principal moment of inertia is preferably at least 10 times smaller, particularly at least 50 times smaller, than the other two principal moments of inertia.

It has also been found that the regions of a metal strip rolled using the process according to the invention have improved resilience by up to a factor of 6 compared with conventionally rolled regions. Strips that have been rolled to a given, constant thickness by the rolling process according to the invention, have a flatter spring characteristic in the rolled region than a strip of the same metal that has been rolled to the same thickness partly by a conventional rolling process with two rolls, i.e. less force is required to obtain a given bending in the strip rolled according to the invention than is the case for a strip having the same thickness but which has been rolled conventionally. Moreover, the spring characteristic in the strip that has been rolled to constant thickness with the process according to the invention is slightly degressive, i.e. with increasing bending less force is required to bend the strip even further. For example, if the strips A and B described previously were clamped at the ends thereof which are 1.5 mm thick, the following deflections were observed upon suspending various weights from the 0.85 mm thick, free ends thereof at a distance of 70 mm from the end (average values from 5 strips each):

TABLE 3

| | Deflection | Deflection |
|---|---|---|
| Suspended weight | (Strip A) | (Strip B) |
| 50 | 0.07 | 0.05 |
| 100 | 0.14 | 0.10 |
| 150 | 0.21 | 0.15 |
| 200 | 0.29 | 0.20 |
| 250 | 0.38 | 0.25 |

The difference between the spring characteristics of strip A that was rolled according to the invention and strip B that was rolled conventionally becomes more pronounced as the strip is rolled more thinly, i.e. as the degree of quenching (the ratio between the height of the strip after rolling and the height of the strip before rolling) becomes smaller. It is possible that this change in spring characteristic is attributable to the earlier described increase in microstructure inhomogeneity in comparison to the not rolled region of the strip, when using the rolling process according to the invention. On the other hand, it is possible to create a leaf spring part with a progressive spring characteristic using the process according to the invention by rolling the required region to a variable thickness.

If strips A and B described in the preceding are cut through at their thicker ends with a rolled thickness of 1.5 mm and at their thinner ends with a rolled thickness of 0.85 mm transversely to the direction of rolling, and these two cut surfaces for each strip are tested for Vickers hardness according to ISO 4516 and ISO 6507/1 using a Leitz Miniload 2 microhardness tester, the following values for Vickers hardness (in MPa) are determined:

TABLE 4

| Measurement no. | | | | |
|---|---|---|---|---|
| (in parentheses | | | | |
| the distance of | | | | |
| the indentation | | | | |
| point of the | Strip B (left | | Strip A (left | |
| indenter from the | column 1.5 mm, | | column 1.5 mm, | |
| strip edge in mm) | right column 0.85 mm) | | right column 0.85 mm) | |
| 1 (1.3) | 182 | 296 | 217 | 310 |
| 2 (2.6) | 181 | 302 | 209 | 308 |
| 3 (3.9) | 180 | 301 | 210 | 307 |
| 4 (5.2) | 189 | 302 | 211 | 308 |
| 5 (6.5) | 192 | 306 | 208 | 305 |

A relatively thick region that has been rolled according to the invention thus has a significantly higher Vickers hardness than a region that has been rolled to the same thickness by conventional means. The difference becomes smaller for thinner rolled strips.

If the metal strip is made from a steel alloy, as a rule a substantial fraction of deformation martensite is evident at the rolled sites, typically in the range from about 5 to about 50 percent by volume of the metal.

In a first preferred embodiment, the metal strip according to the invention is approximately straight and at least a part of its length is rolled in the method according to the invention. In this context, the term “length” is understood to mean the projection of the metal strip onto its said principal inertia axis with the smallest moment of inertia.

In another preferred embodiment, the at least partly rolled metal strip is bent in a U-shape such that it has two legs. Each of these legs has one or more (preferably exactly one) region that bois adjacent to the U-shaped bending point, which is obtainable by the rolling process according to the invention, and which has the properties indicated in the preceding. The length of this preferred, U-shaped metal strip projected onto the principal axis of inertia having the smallest moment of inertia as described previously is preferably about 90 to about 200 mm, particularly preferably about 100 to about 160 mm. The length of the regions of the two legs rolled according to the invention projected onto this principal axis of inertia is preferably about 30 to about 90 mm particularly preferably about 40 to 80 mm. The thickness of the two legs of the U-shaped metal strip for rolling is preferably in the range from about 1 to about 3 mm, preferably in the range from about 1.25 to about 2.75 mm. The thickness of the regions of the two legs rolled according to the invention is preferably in the range from about 0.5 to about 1 mm, particularly preferably in the range from about 0.7 to about 0.9 mm. The degree of deformation φ, calculated according to the formula

$$ϕ = ln ( \frac{{l}_{1}}{{l}_{0}} ) \times 100 \% \qquad ( 7 )$$

wherein l₁ is the thickness of the rolled region of the leg and l₀ is the thickness of the same region before rolling, is preferably in a range from about 50% to about 120%. The U-shaped metal strip may either be bent into the U-shape first, and then a region may be rolled on each leg at the same time in a rolling process according to the invention with two rolls and a support positioned between them. Alternatively, two regions on a starting shaped body that has not yet been bent into a U-shape may first be rolled individually with just one roll in a process according to the invention and a support, and the initial shaped body may then be bent into a U-shape between the two rolled regions. In this case, the support on which rolling is to be performed preferably has a surface contour that exactly matches the inner contour of the initial shaped body that has already been bent into a U-shape including the two regions of the two legs to be rolled. The initial shaped body for rolling may then be placed with total accuracy on the support, so that the two legs to be rolled hang down on the two surface sides of the support. If the two legs are then rolled at the same time, preferably with a rolling device according to the invention equipped with a pair of identical devices each having a roll; and preferably such that the roll device works from top to bottom, the initial shaped body is prevented from slipping during the rolling operation.

The metal strip according to the invention is suitable as an intermediate product in the manufacture of various objects such as were exemplified above. For this purpose, the metal strip may be processed further in subsequent processing steps, such as stamping, drilling, milling, bending, grinding, or even using the rolling process according to the invention to create a desired end product.

If the metal strip according to the invention is straight, it may be processed further to produce for example springs, particularly leaf springs, helical springs or watch springs, or knife blades. In the latter case, it is the knife blade that has been rolled using the process according to the invention.

If the metal strip is bent in a U-shape, as is preferred according to the invention, it may be processed further to produce single part instruments having a gripping function, such as tweezers, tweezer scissors, tongs (for example sugar or ice cube tongs). Tweezer scissors are tweezers on which the two free ends of the legs are shaped into scissor blades, and which slide past each other in a scissor action when the legs are squeezed together. This scissor effect may be directed forwards or backwards. Tweezer scissors with backwardly directed scissor action may be created by fashioning scissor blades on the ends of the legs and then bending the tips of the legs inwards and backwards.

For a tweezers with forward scissor action, the term “forward cutting tweezer scissors” may also be used for the purposes of this invention. For a tweezers with backward scissor action, the term “backward cutting tweezer scissors” may also be used for the purposes of this invention.

The U-shaped bending point makes the tweezers according to the invention, with or without scissor action, easier to sterilise and clean because there is no joining point at the rear end thereof, at which the two legs would meet at an acute angle. This acute, hard-to-reach joint on previously known instruments is where dirt and bacteria can collect. The tweezers with or without scissor action according to the present invention have longer and more resilient legs than the previously known instruments, and thus enable better adjustment of squeezing pressure when closing or releasing the two legs. The weld spot at the rear end of previously known instruments represents a site that is prone to corrosion, and is avoided in the tweezers according to the invention due the single step rolling and bending into a U-shape, with no welding, according to the invention (if a process variant with two paired devices, each furnished with one roll is selected). The ends of the two legs fit together more precisely, so that manual reworking of the legs, as is often necessary with the previously known manufacturing process including welding of the legs, is avoided.

The metal strip according to the invention may also be used to manufacture supporting implants and joint prostheses that are designed to support an impaired joint function. They promote the distraction of the cooperating parts of the joint (support implants, particularly for supporting a hip, knee, or other joint, for example) or replace a lost joint function (joint prostheses).

A common feature of all such support implants or prostheses is that leaf spring components which have been rolled using the process according to the invention ensure mobility in the primary load direction of the joint concerned. Depending on the movement type of the joint, i.e. flexion/extrusion, abduction/adduction, lateral flexion or interior and exterior rotation, one or more such leaf springs are present and are preferably loaded by traction, but may also sustain compression and torsion depending on their design.

Joint prostheses according to the invention may be used to replace a joint entirely, and in general may be adapted to any joint, for example the hip, spine, wrists and ankles, or the mandibular joint. The latter is a preferred example of a joint. The prosthesis may be attached either to the two remaining bone ends of the joint on the flexor side, to the two remaining bone ends on the extensor side, or crossed to one bone end on the flexor side and to the other bone end on the extensor side. In the case of the spine, the element may be attached on both sides in the area of the spinous processes/rib attachment sites.

Supporting implants according to the invention also include, besides the leaf spring components described previously, U-shape bending points that have not been rolled, and in which the anisotropic orientation of the crystallites is consequently again less pronounced. For the purposes of the invention, a “U-shaped” bending point does not necessarily mean that the bending point causes a change of direction through 180°; “U-shaped” typically means a deflection by 90° to 220°, preferably by 160° to 210°, particularly preferably by 170° to 200°, most preferably by 175° to 186°, or by exactly 180°. The leaf spring parts themselves may be flat or may present a certain curvature of constant or variable radius, or a bulging. The leaf spring parts may also be designed as a progressively or partially effective squib to stabilise its extensor effect. In the supporting implants according to the invention, leaf spring parts and bends are preferably arranged in an alternating sequence. Preferred examples of joints that may be supported by the supporting implants according to the invention may be ellipsoid joints (Articulatio ellipsoidea); hinge joints (Ginglymus, for example the finger joints), pivot joints (Articulatio trochoidea, for example the joint between the ulna and the radius); or bicondylar joints (Articulatio bicondylaris, such as the knee joint). Knee joints are a particularly preferred example.

The invention will now be explained in greater detail with reference to the figures. In the figures:

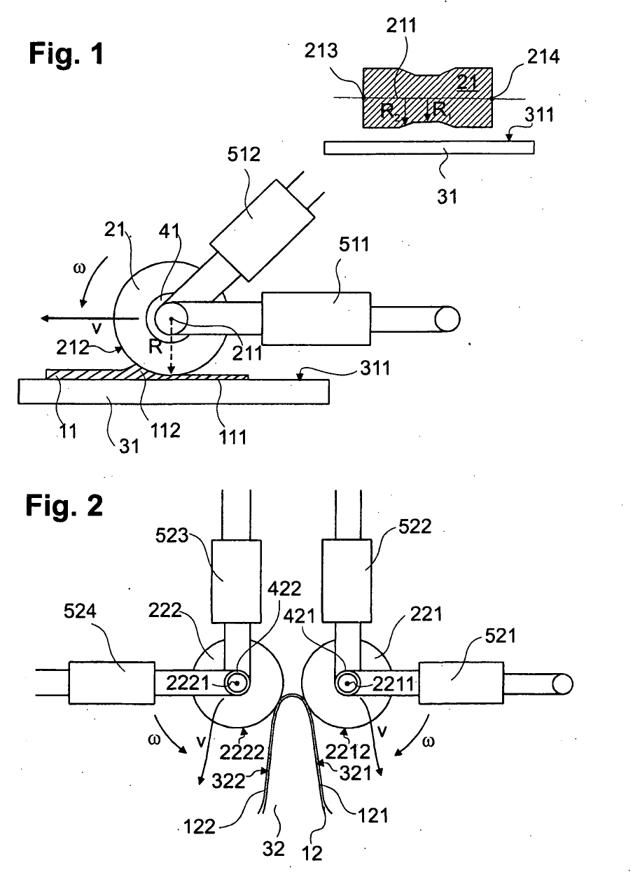

FIGS. 1 and 2 are schematic representations of two variants of the rolling process according to the invention and the associated device;

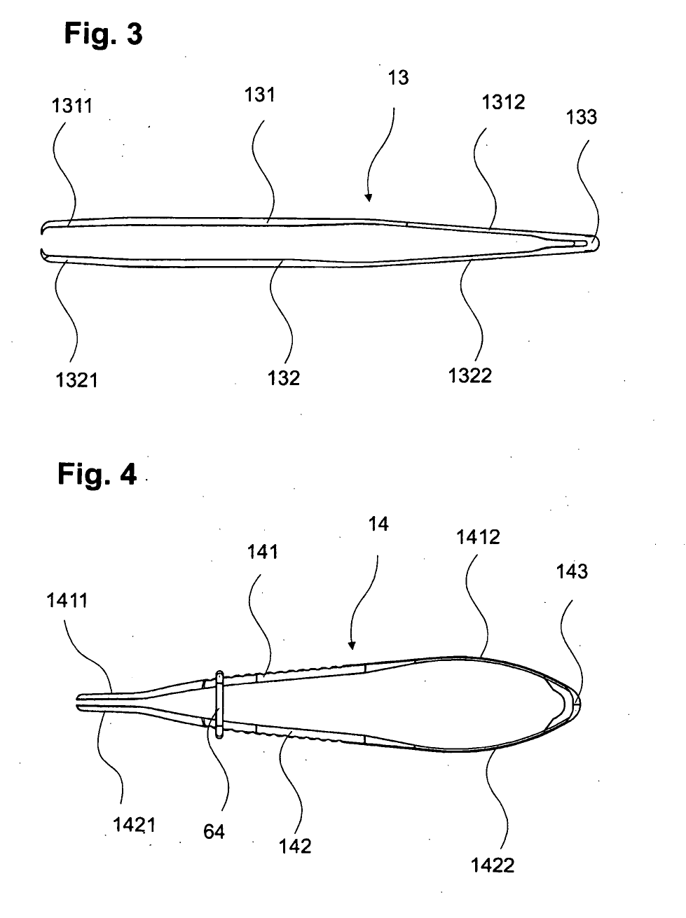

FIGS. 3 to 6 show tweezers and tweezer scissors that are obtainable using the metal strip preferably bent into a U-shape according to the invention as an intermediate product;

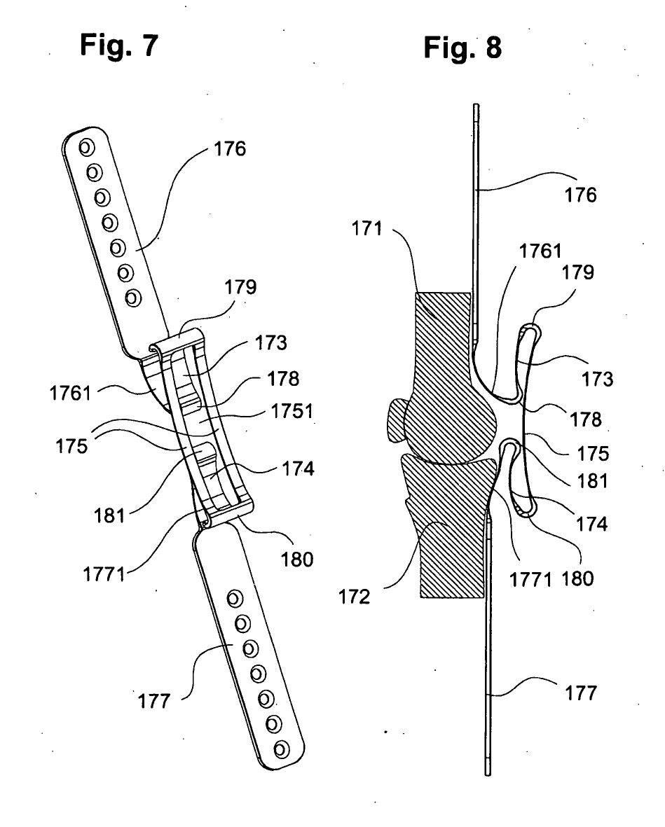

FIGS. 7 and 8 show a supporting implant according to the invention for a knee joint with the knee joint in the extended position;

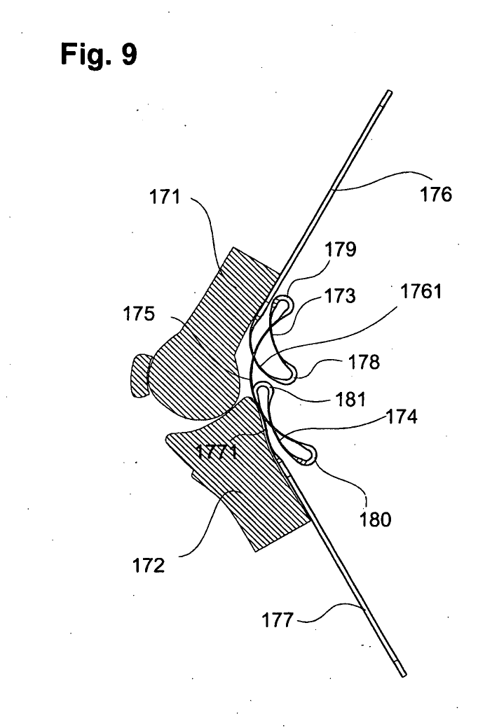

FIG. 9 shows the supporting implant of FIGS. 7 and 8 with the knee in the flexed position; and

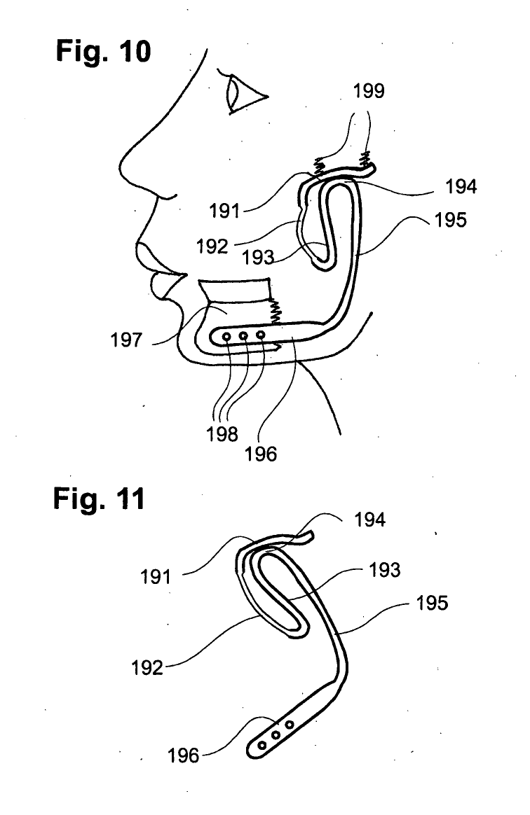

FIGS. 10 and 11 are schematic representations of the function of a mandibular joint prosthesis according to the invention.

In a first preferred embodiment (FIG. 1), the process according to the invention is characterized by just one single, cylindrical roll 21 and a support 31. The initial shaped body 11 is processed to form an at least partially rolled metal strip 111. The figure also shows a distance R, the angular velocity ω and the rolling speed v, as are used in claim 1. The axis of rotation 2111 of the roll is shown only as a point here, because it stands vertically on the leaf plane. The detail at top right in FIG. 1 shows a cross section of the associated roll 21 in the form of a rotationally symmetrical profile roll. The detail shows a distance R₁ from the axis of rotation to a rolling point on the roll surface, which is minimal compared to a second distance R₂ of another rolling point on the roll surface. The detail also shows axis of rotation 211 as a dashed line with the two points 213, 214 at which it passes through the roll 21. Since in this case the support 31 has a flat support surface 311, this normally results in a straight rolling direction v. A friction brake 41, for example a disc brake, is shown in the figure, and is able to exert a braking effect on the angular velocity ω of the roll 21. For the process according to the invention, brake 41 is optional, but for the device according to the invention, it is essential. The figure also shows how a material bulge 112 is formed in front of the roll as a result of the rolling process according to the invention, and helps to brake the roll 21. Two hydraulic guides 511 and 512, which serve to press the roll 21 and thrust it forward, are also shown.

In a second preferred embodiment of the process according to the invention (FIG. 2) exactly two rolls 221, 222, for example of the kind shown in the detail of FIG. 1, are present, and act in rolling manner on the initial shaped body 12 (here bent beforehand into a U-shape having two legs 121, 122) from either side of the support 32. A first rolling operation is performed on the first leg 121 with a first roll 221 and on a first support surface 321; at the same time, another rolling operation is performed on the second leg 122 with a second roll 222 and a second support 322, which faces away from the first support surface 321. Since in this case the support surfaces 321, 322 are no longer flat, the resulting rolling direction v for the two rolls 221, 222 is no longer straight, but curved, reflecting the surface curvature of the support surfaces 321, 322. This may also result in a rolling speed v that is not only no longer straight, but is also variable in terms of its magnitude. The figure shows two friction brakes 421 and 422 (drum brakes, for example), which are able to slow down rolls 221 and 222, respectively. The brakes are optional for the method according to the invention, but essential for the device according to the invention. Each of the two rolls 221 and 222 is also pressed against the initial shaped body 12 and pushed forwards by a pair of hydraulic guides 521, 522 and 523, 524, respectively. The device shown in FIG. 2 might consist of two identical device parts, a first device part having a roll 221, a brake 421 and hydraulic guides 521, 522; and a second device part having a roll 222, a brake 422 and hydraulic guides 523, 524; wherein these device parts are identical in construction and operate in synchronisation with one another.

FIGS. 3 to 6 show embodiments of the tweezers according to the invention with or without scissor action. The common features of all these tweezers are the U-shaped bend 133, 143, 153 and 163 and the two legs 131/132, 141/142, 151/152 and 161/162, respectively in each case, each leg 131, 132, 141, 142, 151, 152, 161 and 162 having a region 1312, 1322, 1412, 1422, 1512, 1522, 1612 and 1622, respectively, that has been rolled in the method according to the invention and which is adjacent to bend 133, 143, 153 and 163, respectively. These common features all result from the metal strip that has been bent into a U-shape, and which was obtained as an intermediate product via a process and device according to FIG. 2. These tweezers, whether with or without scissor action, are all preferably made from a steel alloy.

FIG. 3 shows a tweezers 13 according to the invention. The one leg 131 has a region 1312 that has been rolled according to the process of the invention, and the other leg 132 has a region 1322 that has been rolled according to the process of the invention. The two free ends 1311, 1321 of the two legs 131, 132 are also rolled into points and stamped in the manner of teeth, and the points have subsequently been bent towards one another, so that the teeth are able to engage with each other; the embodiment shown here is thus a surgical tweezers. The bottom free end 1321 has a single tooth, whereas the top free 1311 has two teeth.

FIG. 4 shows tweezers 14 according to the invention in the form of a needle holder. The one leg 141 has a region 1412 that has been rolled according to the process according to the invention, and the other leg 142 has a region 1422 that has been rolled according to the process according to the invention. The two free ends 1411, 1421 of the two legs 141; 142 are bent slightly away from one another so that a tweezers point with a relatively large contact surface is obtained, enabling a needle to be held. This embodiment includes grooves extending transversely on the parts of the legs that have not been rolled, only visible in profile in the figure. These transverse grooves may be used to lock the needle holder 14 in the closed position by means of a retaining ring 64 that encircles both legs 141, 142.

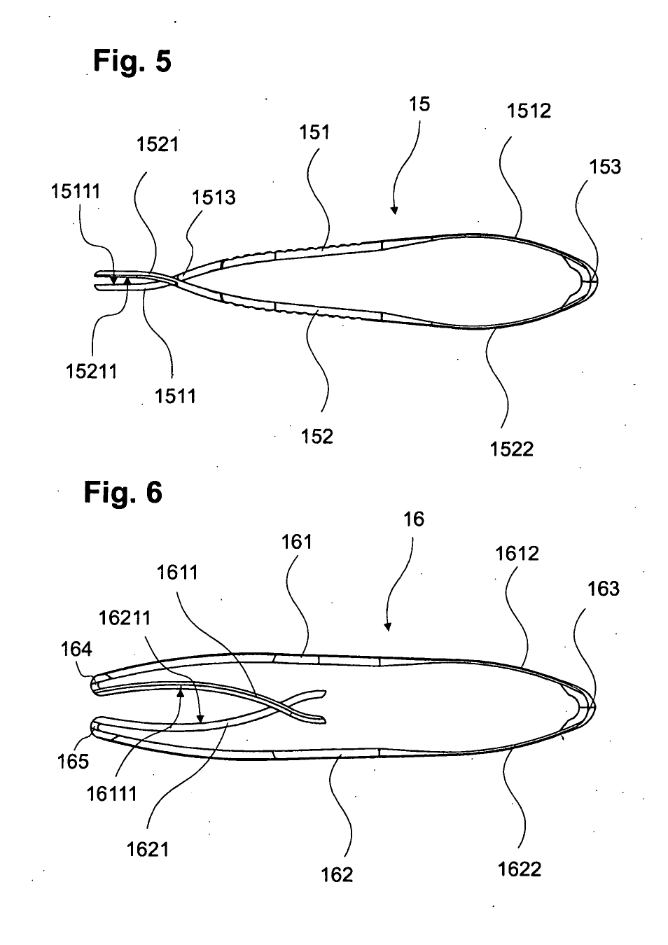

FIG. 5 shows a forward cutting tweezer scissors 15 according to the invention. The one leg 151 has at least one region 1512 that has been rolled according to the process of the invention, and the other leg 152 has at least one region 1522 that has been rolled according to the process of the invention. The free ends 1511 and 1521 cross over one another, and each has a blade 15111 and 15211, respectively. When the two legs 151, 152 are pressed together, leg 152 is levered around a pivot point 1513 provided in first leg 151 (projecting slightly forwards when viewed perpendicularly to the plane of the page), so that the ends 1511, 1521 move towards one another and the blades 15111, 15211 shear against one another in a forwardly moving fashion.

FIG. 6 shows a backwards cutting tweezer scissors 16. The one leg 161 has at least one region 1612 that has been rolled according to the process of the invention, and the other leg 162 has at least one region 1622 that has been rolled according to the process of the invention. The free ends 1611 and 1621 are bent inwards and backwards via a first inversion point 164 and second inversion point 165, respectively, towards the U-shaped bending point 163, and each have a blade 16111 and 16211, respectively, extending from the inversion points 164 and 165, respectively, along the entire length of the ends 1611 and 1621, respectively. When the legs 161, 162 are squeezed together, the inversion points 164, 165 move towards one another and cross over, as a result of which after this point the blades 16111, 16211 also cross over one another and after this point slide past one another backwards in a shearing motion and from that point slide past each other in a shearing manner, progressively backwards towards U-shaped bending point 163, to create a backwards cutting scissor effect.

FIGS. 7 and 8 show a supporting implant for a knee joint according to the invention. When viewed from the side (FIG. 8), the supporting implant is shaped roughly like a flattened version of the Greek letter omega “Ω”. The active part of the supporting implant is in the shape of an assembled C spring, wherein the C spring is formed from three leaf spring parts 173, 174, 175 rolled according to the process of the invention and four U-shaped bends 178, 179, 180 and 181. The leaf spring parts are drawn somewhat less boldly than the other parts of the active component of the supporting implant to show that they are typically made thinner by the rolling process. The joint is represented schematically in FIGS. 7 and 8 by femur 171 and fibula 172 (the patella is also indicated, but without a reference number). The supporting implant also includes two foot elements which are used to attach the supporting implant to the bending side of the knee joint. The first foot element comprises a region 176 that is not usually rolled according to the process of the invention, so that the anisotropic orientation of the crystallites in this region is less pronounced than in the three leaf spring parts 173, 174, 175, and it usually has the form of a plate. The first foot element may be adjoined directly to the first bend 178 or, preferably, via a third, shorter leaf spring part 1761, which provides increased flexibility. The same applies for the second foot element with its region 177 that has not been rolled according to the process of the invention, and which may adjoin a fourth U-shaped bend 181 either directly or, also preferably, via a fourth shorter leaf spring part 1771. In the embodiment shown in FIGS. 7 and 8, the supporting implant is designed for attachment to the bending sides of femur 171 and fibula 172; if the regions 176, 177 that have not been rolled in accordance with the invention are shaped differently from the examples illustrated, the supporting implant might also be attached to another site on the femur 171 and the fibula 172, such as to the extensor side or laterally onto the joint. The three leaf spring parts 173, 174, 175 are connected to each other and to the foot elements via U-shaped bends 178, 179, 180, 181 that have usually not been rolled in the manner of the invention. The first bend 178 connects the first foot element to the first shorter leaf spring part 173; the second bend 179 connects the first shorter leaf spring part 173 to the longer leaf spring part 175; the third bend 180 connects the longer leaf spring part 175 to the second shorter leaf spring part 174, and the fourth bend 181 connects the second smaller leaf spring part 174 to the second foot element. First shorter leaf spring part 173, second U-shaped bend 179, longer leaf spring part 175, third U-shaped bend 180 and second shorter leaf spring part 174 together make up the said active part of the supporting implant in the form of a C spring. The back of this C spring is formed directly by longer leaf spring part 175. The longer leaf spring part 175 has a curvature that is the reverse of the U-shaped curvatures of second U-shaped bend 179 and third U-shaped bend 180, i.e. it is concave, and which faces towards the opening of the C spring. Thus, when attached to the knee, the bend of the C spring is facing towards the knee, in particular towards the hollow of the knee. The longer leaf spring part 175 has a preferably elongated slot or rectangular aperture 1751 that extends in the longitudinal direction of the longer leaf spring part 175 and extends, also preferably, for the entire length thereof. This aperture 1751 becomes important when the knee is bent (see description of FIG. 9 below). To match the asymmetry of the heads of the femur and fibula bones, the overall design of the supporting implant is also asymmetrical.

FIG. 9 shows how the supporting implant of FIGS. 7 and 8 collapses or folds together when the knee is bent. In this event first U-shaped bend 178 and fourth U-shaped bend 181 protrude through aperture 1751 in longer leaf spring part 175, and at least a part of first shorter leaf spring part 173 and at least a part of second shorter leaf spring part 174 do likewise. Conversely, longer leaf spring part 175 bends farther towards the hollow of the knee. The outer edges of the two shorter leaf spring parts 173, 174 and the longer inner sides of aperture 1751 in longer leaf spring part 175 move past each other in a nearly shearing manner. Since the three leaf spring parts 173, 174, 175 and the two further, optional leaf spring parts 1761, 1771 impart the supporting implant a high degree of flexibility in several directions, it might also follow a protrusion or retrusion motion of the joint with lateral approach of first bend 178 and fourth bend 181. By collapsing or folding together in this way, the space occupied by the supporting implant when the knee is bent is comparable to the space it takes up when the knee is extended.

By virtue of its distraction effect, the supporting implant of FIGS. 7-9 guarantees that the joint gap will be kept open in any position of the joint, even under extremely high compressive forces, with the result that the bones associated with the joint are able to move towards each other without contact (sites where the layer of cartilage is missing or partially destroyed due to wear allow bone to rub on bone, which causes the pain). According to the latest information available to the applicant, its shape of a flattened omega character when seen from the side has proven to be the best embodiment for reproducing the motion sequences of a knee joint as faithfully as possible.

FIGS. 10 and 11 show a joint prosthesis according to the invention which is most particularly suitable for use as a mandibular joint prosthesis. It includes an upper jaw part 191, a leaf spring part 192, an ascending leg part 193, a support part 194, a descending leg part 195 and a lower jaw part 196. FIG. 10 shows the mandibular joint prosthesis in the implanted state when the patient's mouth is closed.

In this context, upper jaw part 191 is attached to the remaining part of the upper jaw via screws 199 and lower jaw part 196 is attached to the remaining part of the lower jaw via screws 198. The supporting part 194 fits against the upper jaw part 191 from below 191; supporting part 194 and the underside of upper jaw part 191 together ensure mobility approximating the natural movement of the jaw (rotation and displacement of the lower and upper jaws relative to one another). The leaf spring part 192, which is rolled according to the invention, provides the resilient flexibility that the prosthesis requires for this. FIG. 11 shows the same mandibular jaw prosthesis when the patient's mouth is open. In this event, the leaf spring part 192 bends slightly backwards and extends a little, and the supporting part 194 rotates downwards together with descending leg part 195 and lower jaw part 196, whereby the supporting part 194 is able to slightly slide forwards along the underside of the upper jaw part 191. This mandibular jaw prosthesis prevents excessive distraction movements of the upper and lower jaws: The prevention of such distraction begins when the leaf spring part 192 is almost completely straight.

1. A single-piece metal strip having no weld seams and made of a polycrystalline metal, comprising at least one region in which the crystallites have a comparatively more anisotropic orientation, and at least one region in which the crystallites have a comparatively less anisotropic orientation; and wherein θ-2θ-ray diffractograms measured at two arbitrary points of the strip using CuKα radiation show no statistically significant differences with respect to the position or shape between the corresponding pikes.

2. The metal strip of claim 1, characterised in that it is made of a metal that crystallizes according in a body-centred cubic (bcc) crystal lattice, preferably of a ferritic, martensitic, or mixed ferritic/martensitic steel, more preferably of a steel having a composition Cr 12.50-14.50 percent by weight, C 0.42-0.50 percent by weight, Si max. 1.00 percent by weight, Mn max. 1.00 percent by weight, P max. 0.045 percent by weight, S max. 0.030 percent by weight, the rest being essentially iron and unavoidable contaminants.

3. The metal strip of claim 2, characterized in that it has a flat surface and the (200) crystal plane families of the crystallites in the region in which the crystallite orientation is comparatively more anisotropic are more frequently parallel to this surface than the (200) crystal plane families of the crystallites in the region in which the crystallite orientation is comparatively less anisotropic.

4. The metal strip of any of claims 1 to 3, characterised in that the region in which the crystallite orientation is comparatively more anisotropic also has a microstructure with comparatively more pronounced inhomogeneity than the region in which the crystallite orientation is comparatively less anisotropic.

5. A rolling process for shaping a metallic initial shaped body (11, 12), wherein the rolling operation wherein the rolling process is performed between a roll (21, 221, 222) having an axis of rotation (211, 2211, 2221) and a rolling surface (212, 2212, 2222) on the one hand, and a support (31, 32) having a support surface (311, 321, 322) on the other hand; characterized in that the angular velocity ω of the roll is controlled in such a manner that
$$0 \leq ω < \frac{v}{R} \qquad ( 1 )$$
applies for at least one point of the roll surface (212, 2212, 2222) that contacts the initial shaped body (11, 12) in rolling manner, and in which ν is the rolling velocity and R is the distance between the axis of rotation (211, 2211, 2221) and the said point on the roll surface (212, 2212, 2222) measured perpendicularly to the axis of rotation (211, 2211, 2221) of the roll; by which rolling process a metal strip according to any one of claims 1 to 4 is or may be obtained, in which the region rolled in this manner is the region of relatively more pronounced anisotropic crystallite orientation, and in which a not rolled region is the region of relatively less anisotropic crystallite orientation.

6. The process of claim 5, characterised in that the angular velocity ω of the roll is controlled in such manner that
$$0 \leq ω < \frac{v}{R} , \qquad ( 1 )$$
applies for each point of the roll surface (212, 2212, 2222) that contacts the initial shaped body (11, 12) in rolling manner, wherein ω, v and R mean the same as in claim 1.

7. The process of claim 5 or 6, characterised in that the initial shaped body (12) is bent in a U-shape such that it has two legs (121, 122), that the support (32) has a first support surface (321) and a second support surface (322), that the first leg (121) is rolled between a first rolling surface (2212) of a first roll (221) and the first support surface (321), and at the same time the second leg (122) is rolled between a second rolling surface (2222) of a second roll (222) and the second support surface (322).

8. The process of one of claims 5 to 7, characterised in that the angular velocity ω of each roll (21, 221, 222) is in the range of 30 to 95%, preferably 50 to 80% of the quotient v/R.

9. The process of one of claims 5 to 8, characterised in that braking of the roll or rolls (21, 221, 222) is effected with a friction brake, for example a disc brake (41) or a drum brake (421, 422) or by means of an eddy current brake, or that the angular velocity of the roll is controlled via the rotating speed on an electric or hydraulic motor.

10. The process of one of claims 5 to 9, characterised in that the initial shaped body (11, 12) consists of a steel alloy, preferably of a ferritic, martensitic, or mixed ferritic/martensitic steel, more preferably of a steel having a composition Cr 12.50-14.50 percent by weight, C 0.42-0.50 percent by weight, Si max. 1.00 percent by weight, Mn max. 1.00 percent by weight, P max. 0.045 percent by weight, S max. 0.030 percent by weight, the rest being essentially iron and unavoidable contaminants.

11. The process of one of claims 5 to 10, characterized in that the roll is a profile roll.

12. A rolling device for performing the process of claim 5, comprising a roll (21, 221, 222) with an axis of rotation (211, 2211, 2221) and a rolling surface (212, 2212, 2222), a support (31, 32) with a support surface (311, 321, 322) and a brake (41 or 421 or 422), which is able to brake the roll (21 or 221 or 222) during rolling.

13. The device of claim 12, characterised in that it comprises a first roll (221) with a first axis of rotation (2211) and a first rolling surface (2212), a first brake (421), a second roll (222) with a second axis of rotation (2221) and a second rolling surface (2222), a second brake (422) and a support (32) with a first support surface (321) and a second support surface (322); that the first roll (221) is able to perform a rolling action on the first support surface (321) and the second roll (222) is able to perform a rolling action on the second support surface (322) at the same time; and that the first brake (421) is able to brake the first roll (221) and the second brake (422) is able to brake the second roll (222).

14. A single-piece tweezers (13, 14, 15, 16) having no weld spot, with a U-shaped bending point (133, 143, 153, 163) and two resilient legs (131/132, 141/142, 151/152, 161/162), wherein each leg (131, 132, 141, 142, 151, 152, 161 and 162) has a distal end (1311, 1321, 1411, 1421, 1511, 1521, 1611 and 1621, respectively), characterised in that the legs (131, 132, 141, 142, 151, 152, 161 and 162, respectively) each have at least one region (1312, 1322, 1412, 1422, 1512, 1522, 1612 and 1622, respectively), which is obtainable according to the rolling process of one of claims 5 to 11, or in which the crystallite orientation is comparatively more anisotropic; the bending point of the tweezers (133, 143, 153, 163) is a region in which the crystallite orientation is comparatively less anisotropic, and θ-2θ-ray diffractograms measured at two arbitrary points of the tweezers using CuKα radiation show no statistically significant differences with respect to the position or shape between the corresponding pikes.

15. The tweezers (15) of claim 14, characterised in that the distal ends (1511 or 1521) have a blade (15111 and 15211, respectively) and that the first leg (151) has a pivot point (1513) designed such that when the legs (151, 152) are squeezed together the second leg (152) is able to rock over the pivot point (1513), the ends (1511, 1521) are able to move towards one another, and the blades (15111, 15211) are able to slide past one another in a shearing manner during this approaching motion.

16. The tweezers (16) of claim 14, characterised in that each of the distal ends (1611 and 1621) is bent inwards and backwards towards the U-shaped bend point (163) via an inversion point (164 and 165, respectively), wherein each end (1611 and 1621) is provided as from the inversion point (164 and 165, respectively) with a blade (16111 and 16211, respectively) such that when the legs (161, 162) are pressed together the inversion points (164, 165) are able to move towards and cross over one another, and after the inversion points (164, 165) cross over one another the blades (15111, 15211) are able to slide past one another progressively backward in a shearing manner.

17. The tweezers of one of claims 14 to 16, consisting of a steel alloy, preferably of a ferritic, martensitic or mixed ferritic/martensitic steel, more preferably of a steel having a composition Cr 12.50-14.50 percent by weight, C 0.42-0.50 percent by weight, Si max. 1.00 percent by weight, Mn max. 1.00 percent by weight, P max. 0.045 percent by weight, S max. 0.030 percent by weight, the rest being essentially iron and unavoidable contaminants.

18. A supporting implant for a joint, comprising a distal and a proximal bone end, wherein these bone ends are connected to one another in articulated manner, and wherein the supporting implant comprises a metal strip according to one of claims 1 to 4, or consists thereof.

19. The supporting implant of claim 18 for a knee joint, wherein the metal strip contains three regions in the form of three leaf spring parts (173, 174, 175) in which the crystallite orientation is comparatively more anisotropic, or which are obtainable via the rolling process of one of claims 5 to 11, the metal strip includes four U-shaped bends (178, 179, 180, 181) and two regions in the form of two foot elements (176/1761, 177/1771), each of which includes a region (176, 177) in which the crystallite orientation is comparatively less anisotropic or that has not been rolled; wherein
a) a first foot element (176/1761) adjoins a first U-shaped bend (178), a first, shorter leaf spring part (173) adjoins the first U-shaped bend (178), a second U-shaped bend (179) adjoins the first shorter leaf spring part (173), a longer leaf spring part (175) with an elongated, particularly slot-like or rectangular aperture (1751) conformed therein adjoins the second U-shaped bend (179), a third U-shaped bend (180) adjoins the longer leaf spring part (175) a second shorter leaf spring part (174) adjoins the third U-shaped bend (180), a fourth U-shaped bend (181) adjoins the second leaf spring part (174), and a second foot part (177/1771) adjoins the fourth U-shaped bend (181); such that first shorter leaf spring part (173), second U-shaped bend (179), longer leaf spring part (175), third U-shaped bend (180) and second shorter leaf spring part (174) together form a C spring, the back of which is formed by the longest leaf spring part (175) and is curved towards the opening of the C spring, and the apertures of the first (178) and fourth U-shaped bends (181) face away from the opening of the C spring, so that the two foot elements (176/1761, 177/1771), which adjoin the first (178) and fourth (181) U-shaped bends, are spread away from the opening of the C spring,
b) the supporting implant is attachable to the femur (171) via one of the two foot elements (176/1761), and to the fibula (172) via the other of the two foot elements (177/1771) such that the C spring lies flush against the bending side of the knee joint and the opening of the C spring faces towards the knee joint, and
c) after the supporting implant has been attached to the knee the back of the C spring is able to bend further towards the knee when the knee is bent, and the two assemblies of first U-shaped bend (178) and first, shorter leaf spring part (173) on the one hand and of third U-shaped bend (180) and second shorter leaf spring part (174) on the other hand are able to protrude at least partially thought the aperture (1751) when the knee is bent.

20. The supporting implant of claim 19, characterised in that the region (176) of comparatively less anisotropic crystallite orientation of the first foot element adjoins the first U-shaped bend (178) via a third shorter leaf spring part (1761), and the region (177) of comparatively less anisotropic crystallite orientation of the second foot element adjoins the fourth U-shaped bend (181) via a fourth leaf spring part (1771).

21. A joint prosthesis for complete replacement of a joint, comprising or consisting of a metal strip of one of claims 1 to 4.

22. The joint prosthesis as recited in claim 21 for complete replacement of a mandibular joint, wherein the metal strip has a region in the form of a leaf spring part (192) which has comparatively more anisotropic crystallite orientation or which is obtainable using the rolling process of one of claims 5 to 11, and five regions preferably with comparatively less anisotropic crystallite orientation in the form of an upper jaw part (191), an ascending leg part (193), a support part (194), a descending leg part (195) and a lower jaw part (196); such that the leaf spring part (192) adjoins the upper jaw part (191), the ascending leg part (193) adjoins the leaf spring part (192), the support part (194) adjoins the ascending leg part (193), the descending leg part (195) adjoins the support part (194), and the lower jaw part (196) adjoins the descending leg part (195); and such that leaf spring part (192) and ascending leg part (193) form a first loop, the opening of which faces towards the upper jaw part (191), and ascending leg part (193), support part (194) and descending leg part (195) form a second loop, the opening of which faces in the opposite direction; support part (194) and upper jaw part (191) are able to contact one another in such manner that the support part (194) is able to roll over the upper jaw part (191) and slide along it, so that due to the leaf spring part (192), the point of contact between the upper jaw part (191) and the support part (194) the prosthesis has the movement capabilities of a mandibular joint.
